(19) United States
(12) Patent Application Publication
(10) Pub. No.: US 2018/0279951 A1
(43) Pub. Date: Oct. 4, 2018

(54) MOVEMENT COMPENSATION FOR SENSOR-EQUIPPED ATHLETIC GARMENTS

(71) Applicant: Mad Apparel, Inc., Redwood City, CA (US)

(72) Inventors: Anna Asnis, San Jose, CA (US); James Artel Berg, Redwood City, CA (US); Hamid Hameed Butt, Cupertino, CA (US)

(21) Appl. No.: 15/478,070

(22) Filed: Apr. 3, 2017

(51) Int. Cl. A61B 5/00; A41D 1/00; A41D 13/00; A41D 31/02; A61B 5/0245; A61B 5/0488; A63B 24/00

(52) U.S. Cl. CPC A61B 5/6804; A41D 1/002; A41D 13/0015; A41D 31/02; A61B 5/6805; A61B 2503/10; A61B 5/0488; A61B 5/0022; A61B 5/486; A63B 24/0062; A61B 5/0245

(57) ABSTRACT

An athletic garment includes connective segments that compensate for motion of an athlete wearing the athletic garment. Right and left connective segments couple right and left arm segments of the garment to a torso segment, respectively. Thus, the connective segments may overlay the armpit areas of the athlete. The connective segments are composed of an elastic material such that as the athlete raises the arms of the athlete, there is minimal or no shifting of the torso segment. Further, the garment includes sensors that contact the skin of the athlete and generate physiological data such as activation levels of the athlete's muscles. Due to the movement compensation provided by the connective segments, sensors located on the torso segment may not shift as much while the athlete is performing exercises, which can allow the sensors to generate more reliable and accurate physiological data.

### BACKGROUND

### 1. Field of Art

This description generally relates to sensor-equipped athletic garments, and specifically to compensating for movement of an athletic garment worn by a user while the user is exercising.

### 2. Description of the Related Art

Garments can include sensors that record a variety of information about the human body. For example, electrocardiograph (ECG) electrodes can measure electrical signals from the skin of a person that are used to determine the person's heart rate. In addition, electromyography (EMG) electrodes can measure electrical activity generated by a person's muscles. Heart rate and muscle movement information may be useful for evaluating the person's physiological condition, for instance, while exercising.

While an athlete wearing a conventional garment is exercising, the conventional garment may shift in position relative to the athlete's body. For example, if the athlete raises the athlete's arms, a torso segment of the conventional garment also shifts up. Thus, any sensors such as an ECG or EMG electrode located on the torso segment will also shift in position, resulting in unreliable or inaccurate data for the athlete. This issue is particularly prevalent for garments composed of compressive material such as Spandex because these garments are held tightly against the athlete's body. It is desirable and challenging to develop a sensor-equipped athletic garment that compensates for motions of an athlete wearing the athletic garment.

### SUMMARY

An athletic garment includes connective segments that compensate for motion of an athlete wearing the athletic garment. Right and left connective segments couple right and left arm segments of the garment to a torso segment, respectively. Thus, the connective segments may overlay the armpit areas of the athlete. Further, the garment includes sensors that contact the skin of the athlete and generate physiological data such as activation levels of the athlete's muscles. The sensors may be positioned on both the right and left arm segments and the torso segment. The sensors are electrically coupled via conductive threads (embedded in the athletic garment) to a mount that interfaces with a processing unit. The processing unit can process physiological data received from the sensors to generate biofeedback for the athlete, for example, describing the athlete's exertion of a particular muscle while exercising.

The connective segments are composed at least of an elastic material such that as the athlete raises the arms of the athlete, the connective segments stretch, minimizing any shifting of the torso segment relative to the torso of the athlete. For instance, the elastic material has an elastic modulus that is less than the elastic modulus of the material of the arm and torso segments. Thus, for a given threshold of motion (e.g., lateral deformation or rotation), the movement of the arm segments does not cause reciprocal movement of the torso segment. Due to the movement compensation provided by the connective segments, the sensors may generate more reliable and accurate physiological data.

In some embodiments, the athletic garment is not necessarily a shirt with arm segments and a torso segment. For example, the athletic garment is a pants or shorts garment with sensors positioned on leg segments of the athletic garment. In one embodiment, a garment comprises a conductive thread coupled within a fabric layer. The garment further comprises a first segment composed of at least a first material having a first elastic modulus. The garment further comprises a connective segment composed of at least a second material having a second elastic modulus less than the first elastic modulus. The connective segment is coupled to the first segment and configured to overlay a portion of an athlete wearing the garment. The garment further comprises a second segment coupled to the connective segment.

In some embodiments, the athletic garment includes at least an inner layer and an outer layer. The inner layer includes the sensors of the garment and may expose the armpit areas of the athlete to provide movement compensation. The outer layer is composed of at least a compressive material to maintain the position of the inner layer relative to the athlete's body. In some embodiments, the athlete may wear a sleeveless garment over the athletic garment to maintain the position of the athletic garment relative to the athlete's body.

### BRIEF DESCRIPTION OF DRAWINGS

FIG. 1A shows an inside view of a front side of an athletic garment according to one embodiment.

FIG. 1B shows an inside view of a back side of the athletic garment shown in FIG. 1A according to one embodiment.

FIG. 2A is a diagram of an athlete wearing a conventional garment according to one embodiment.

FIG. 2B is a diagram of the athlete shown in FIG. 2A with raised arms according to one embodiment.

FIG. 3A is a diagram of an athlete wearing the athletic garment shown in FIG. 1A according to one embodiment.

FIG. 3B is a diagram of the athlete shown in FIG. 3A with raised arms according to one embodiment.

FIG. 4A shows an inner layer of an athletic garment according to one embodiment.

FIG. 4B shows an outer layer overlaid on the inner layer of the garment shown in FIG. 4A according to one embodiment.

FIG. 5A shows an outside view of the front side of the athletic garment shown in FIG. 1A according to one embodiment.

FIG. 5B shows an outside view of the back side of the athletic garment shown in FIG. 1A according to one embodiment.

FIG. 6A shows a front side of a sleeveless garment overlaid on the athletic garment shown in FIG. 1A according to one embodiment.

FIG. 6B shows a back side of the sleeveless garment overlaid on the athletic garment shown in FIG. 6A according to one embodiment.

FIG. 7A shows a front side of another sleeveless garment overlaid on the athletic garment shown in FIG. 1A according to one embodiment.

FIG. 7B shows a back side of the sleeveless garment overlaid on the athletic garment shown in FIG. 7A according to one embodiment.

FIG. 8A shows an front view of a pants athletic garment according to one embodiment.

FIG. 8B shows a back view of the pants athletic garment shown in FIG. 8A according to one embodiment.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. Example Sensor-Equipped Athletic Garment

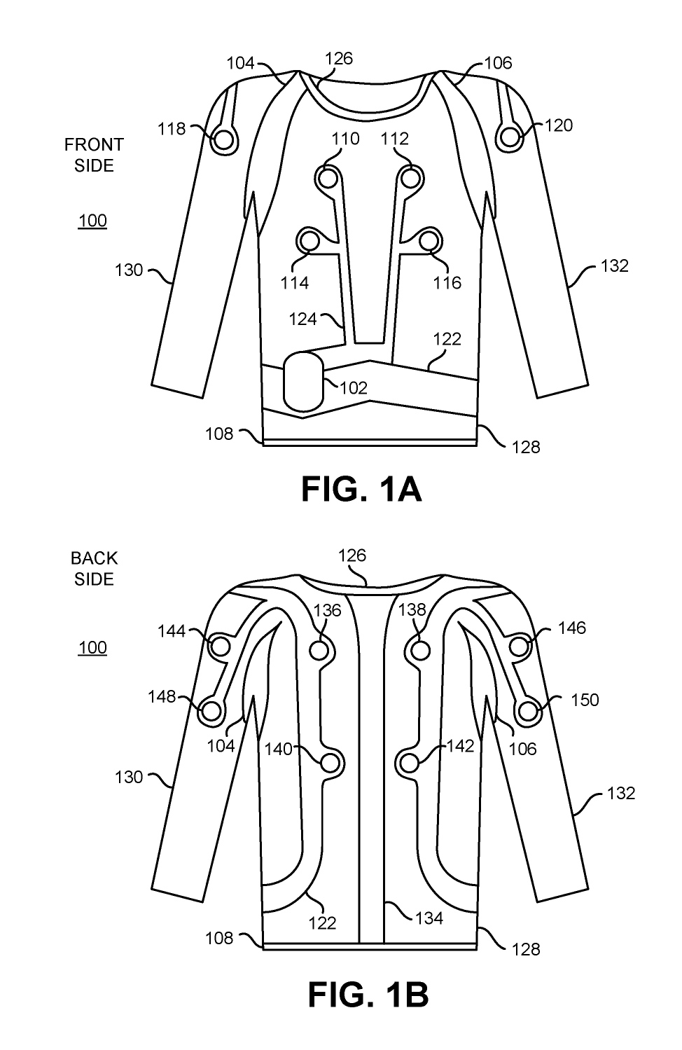

FIG. 1A shows an inside view of a front side of an athletic garment 100 according to one embodiment. FIG. 1B shows an inside view of a back side of the athletic garment 100 shown in FIG. 1A according to one embodiment. An athlete wears the athletic garment 100 while performing exercises. The athletic garment 100 may record physiological data (e.g., muscle activation data or heart rate data) of the athlete using one or more sensors included in the athletic garment 100. For example, the sensors can be electrodes that measure electromyography (EMG) signals (electrical signals caused by muscle cells) also referred to as muscle activation data or electrocardiograph (ECG) signals (electrical signals caused by depolarization of the user's heart muscle in particular) also referred to as heart rate data. The sensors may also include other types of sensors such as accelerometers and gyroscopes (which generate motion data based on the athlete's movement), temperature sensors, pressure sensors, humidity sensors, etc. The sensors generate physiological data of the athlete based on the measured signals, which may be used to generate biofeedback associated with exercises that the athlete performs while wearing the athletic garment 100. The sensors can physically contact the skin of the athlete because the sensors shown in FIG. 1A are located on an inside layer of the athletic garment 100.

The athletic garment 100 includes a torso segment 128, a right arm segment 130, and a left arm segment 132. The torso segment 128 is coupled to a right connective segment 104 and a left connective segment 106. The right arm segment 130 and left arm segment 132 are coupled to the right connective segment 104 and left connective segment 106, respectively. In addition, at least a portion of the right arm segment 130 and left arm segment 132 may also be coupled to the torso segment 128. The athletic garment 100 also includes a collar segment 126, which may be coupled to the left and right connective segments 104 and 106 (or other connective segments) in some embodiments. As shown in FIG. 1A, the right arm segment 130 and left arm segment 132 are not coupled to the torso segment 128 on the front side of the athletic garment 100; however, FIG. 1B shows that the right arm segment 130 and left arm segment 132 are coupled to the torso segment 128 on the back side of the athletic garment 100. In other embodiments, the right arm segment 130 and the left arm segment 132 can be coupled to the torso segment 128 on the front side of the athletic garment 100 as well.

The right connective segment 104 and left connective segment 106 shown in FIG. 1A overlay the right and left armpit areas of an athlete when the athlete wears the athletic garment 100. The athletic garment 100 may also include any number of additional connective segments that overlay with other areas of the athlete's body. For example, connective segment 134 on the back side of the athletic garment 100 shown in FIG. 1B overlays with the spine area of the athlete's body. Further, the athletic garment 100 may include one or more elastic bands such as elastic band 108 (e.g., a compression belt) of the torso segment 128 that overlays the waist area of an athlete wearing the athletic garment 100. In some embodiments, the athletic garment 100 may include a compression belt with a buckle or snap locking mechanism such that an athlete may conveniently secure or loosen the compression belt to wear or take off the athletic garment 100.

Connective segments of the athletic garment 100 are composed of at least an elastic material. For instance, the elastic material has a first elastic modulus, while the right arm segment 130, left arm segment 132, and torso segment 128 are composed of at least another material having a second elastic modulus greater than the first elastic modulus. The elastic modulus represents a resistance of a material to stretch when acted on by a force, in some embodiments. Thus, for a given force applied on the different segments of the athletic garment 100, the resulting deformations of the connective segments are greater than the resulting deformations of the right arm segment 130, left arm segment 132, or torso segment 128. In other words, the connective segments are more “stretchy” than the other three segments. Example elastic materials include synthetic fibers such as Spandex, Lycra, or elastane, stretch vinyl, nylon, polyester and polyamide or some combination thereof. Example material for the right arm segment 130, left arm segment 132, torso segment 128, or collar segment 126 include fabrics such as cotton, nylon, polyester, polypropylene, polyamide, elastane, Spandex, synthetic fibers, or some combination thereof. In addition to material, the elasticity of segments of the athletic garment 100 may be based on the manufacture or construction of the segments. In one embodiment, the connective segments 104 and 106 are composed of 77% nylon and 23% Spandex, and the torso segment 128 is composed of 72% polyamide fabric and 28% elastane.

In some embodiments, connective segments may be composed of multiple types of materials having different elastic moduli. For example, a connective segment is composed of a first material having a first elastic moduli and a second component having a second elastic moduli greater than the first elastic moduli. The second elastic moduli may be less than, equal to, or greater than the elastic moduli of a material of the non-connective segments of the athletic garment 100 (e.g., the right arm segment 130, left arm segment 132, torso segment 128, or collar segment 126). The connective segment may be composed of multiple sub-segments such as an alternating pattern of sub-segments having the first and second elastic moduli. Further, the connective segment may include perforations to adjust its elastic modulus or to provide air flow through the athletic garment 100, or in other words, help the athletic garment 100 become more “breathable,” and thus help facilitate the respiration of an athlete.

In the embodiment shown in FIG. 1A, the athletic garment 100 includes six sensors on the front side of the athletic garment 100 to record muscle activation data from the athlete's muscles nearby each sensor. In particular, sensors 118 and 120 located on the right and left shoulder of the athletic garment 100 can record muscle activation data of the athlete's deltoid muscles. Sensors 110 and 112 located on the right and left chest of the athletic garment 100 can record muscle activation data of the athlete's pectoral muscles. Sensors 114 and 116 located on the right and left abdomen of the athletic garment 100 can record muscle activation data of the athlete's abdominal (also referred to as “abs”) and oblique muscles. Though the athletic garment 100 shown in FIG. 1A includes six sensors on the front side, in other embodiments, the athletic garment 100 can include any number of sensors or other types of components or electronics at any location or configuration within the athletic garment 100.

In the embodiment shown in FIG. 1B, the athletic garment 100 includes eight sensors on the back side of the athletic garment 100 to record muscle activation data from the athlete's muscles nearby each sensor. In particular, sensors 144, 146, 148, and 150 located on the right and left upper arms of the athletic garment 100 can record muscle activation data of the athlete's triceps muscles. Sensors 136 and 138 located on the right and left back side of the athletic garment 100 can record muscle activation data of the athlete's trapezius muscles (also referred to as “traps”). Sensors 140 and 142 located on the right and left back side of the athletic garment 100 can record muscle activation data of the athlete's Latissimi dorsi muscles (also referred to as “lats”).

The sensors of the athletic garment 100 are electrically coupled to a mount 102 via one or more conductive threads. In the example shown in FIG. 1A, sensors 110, 112, 114, and 116 on the front side of the athletic garment 100 are electrically coupled to the mount 102 via a first set of conductive threads 124. In addition, sensors 118 and 120, as well as the eight sensors on the back side of the athletic garment 100 are electrically coupled to the mount 102 via a second set of conductive threads 122. Each set of conductive threads includes one or more conductive threads each coupled to at least one sensor. For instance, the first set of conductive threads 124 includes four conductive threads that are each coupled to one of the sensors 110, 112, 114, and 116. Conductive threads in a set may not be electrically coupled to each other, and thus can transmit different electrical signals.

The conductive thread can be embedded between different fabric layers of the athletic garment 100, for example, to insulate, waterproof, or protect the conductive thread from damage. The conductive thread may be embroidered to a layer of the athletic garment 100, bonded to a layer of the athletic garment 100 using an adhesive, or coupled to the athletic garment 100 using other suitable methods. The conductive threads in one embodiment are twisted nylon threads coated to have a metallic surface. In another embodiment, the conductive paths can be formed using printed conductive resin (e.g., polymers, silicone, neoprene, thermoplastics, etc.), conductive metal (e.g., copper), or any other type of conductive material. The conductive resin is produced by combining conductive material with a resin, for example, a non-conductive resin. In some embodiments, the conductive threads do not overlap or cross within the athletic garment 100. In some embodiments, the conductive thread is less than 1 millimeter thick. As shown in FIGS. 1A-B, the conductive threads are routed through the right arm segment 130, left arm segment 132, and torso segment 128, though in other embodiments, the conductive threads may also be routed through the connective segments or other portions of the athletic garment 100.

The sensors can be communicatively coupled to a processing unit (not shown in FIG. 1A) that is physically coupled to the mount 102. The processing unit can process, aggregate, and analyze the physiological data received from the sensors. The processing unit can also provide the physiological data, analysis, or other biofeedback to a client device via a network (e.g., the Internet, BLUETOOTH®, or WiFi). For example, the client device is a mobile phone, tablet, or computer of the athlete wearing the athletic garment 100 or a coach or friend of the athlete.

In some embodiments, the mount 102 includes one or more mount terminals exposed on an outside surface of the athletic garment 100 that are electrically conductive, and are configured to output signals from sensors of the athletic garment 100 to the processing unit. For instance, the processing unit also includes electrically conductive terminals that interface with the mount terminals. The mount 102 shown in FIG. 1A is located on the bottom right of the front side of the torso segment 128, though in other embodiments, the mount 102 may be located in any suitable position of the athletic garment 100, e.g., on the back side of the torso segment 128 or one of the arm segments.

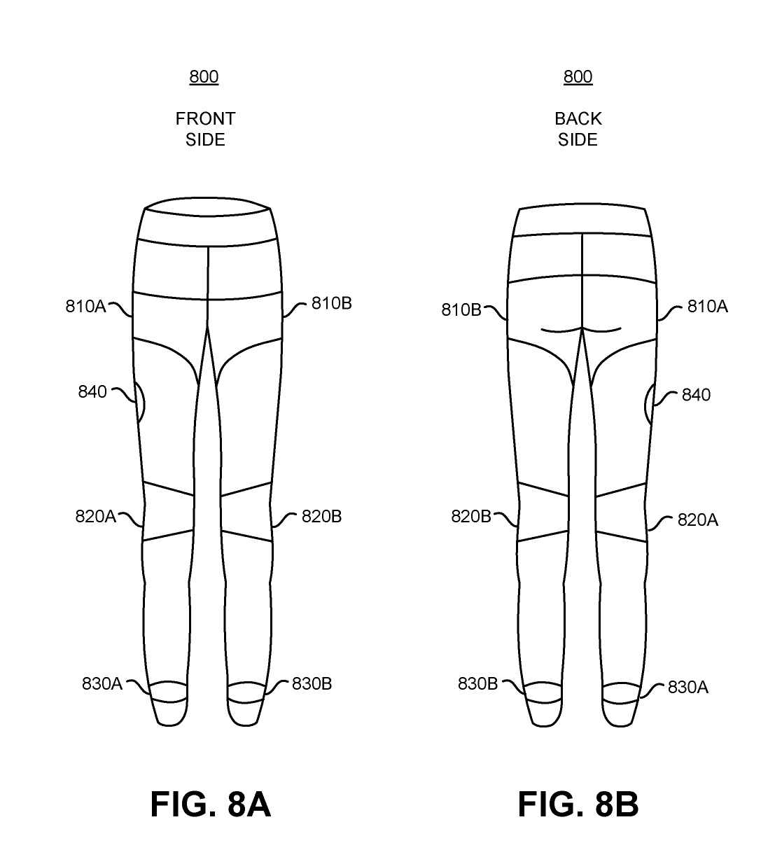

It should be noted that while the athletic garment 100 shown in FIGS. 1A-B is a long sleeve shirt, the principles described herein apply equally to any garment, including but not limited to a short sleeved shirt, a tank top, pants, shorts, or any other suitable garment. In embodiments where the athletic garment 100 is a pair of pants or shorts, sensors of the athletic garment 100 can record muscle activation data from muscles on an athlete's lower body, e.g., quadriceps, glutes, hamstrings, calves, and the like. An example pants athletic garment is described below with reference to FIGS. 8A-B. In other embodiments, one or more connective segments of an athletic garment 100 may overlay portions of the athlete's body other than the armpit or spine area. For example, connective segments may be incorporated in the athletic garment 100 to overlay the area behind the athlete's knees (e.g., the popliteal fossa, also referred to herein as a “knee pit”), the athlete's buttocks, groin region, or any other suitable area of the athlete's body.

### II. Movement Compensation for Athletic Garments

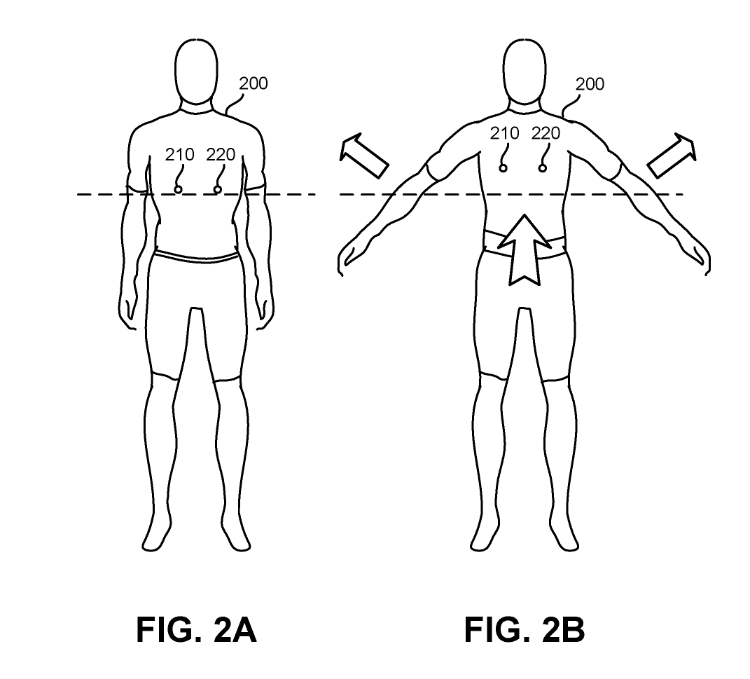

FIG. 2A is a diagram of an athlete wearing a conventional garment 200 according to one embodiment. The conventional garment 200 includes sensor 210 and sensor 220 located on the athlete's abdominal area similar to sensors 114 and 116 of the athletic garment 100 shown in FIG. 1A.

FIG. 2B is a diagram of the athlete shown in FIG. 2A with raised arms according to one embodiment. Since the conventional garment 200 does not compensate for the motion of the raised arms, the torso segment of the conventional garment 200 shifts upward in the direction of the raised arms, which is illustrated by the dotted lines in FIGS. 2A-B. Additionally, the sensor 210 and sensor 220 shift upward as well because the sensors are attached to the torso segment of the conventional garment 200. In other words, motion of the raised arms causes a reciprocal movement of the torso segment, and in extension, a reciprocal movement of the sensors 210 and 220 as well. The shifting of the sensor positions is undesirable because the shifted sensors may no longer be physically contacting a target muscle (e.g., the abs) for generating corresponding physiological data. The shifting can also reduce the contact quality of a sensor with a target muscle, which may result in unreliable or inaccurate physiological data from the sensor. Further, the shifting may decrease the signal-to-noise ratio of the sensor data.

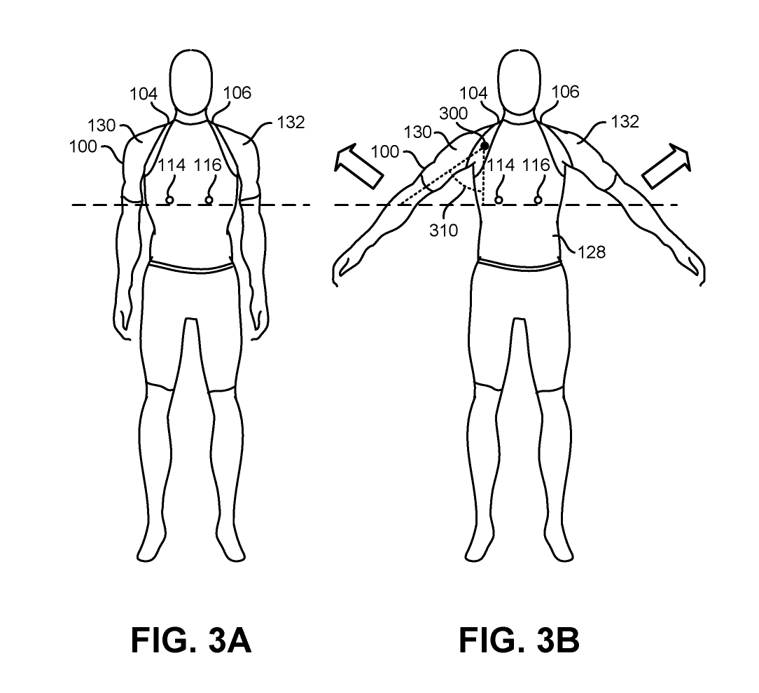

FIG. 3A is a diagram of an athlete wearing the athletic garment 100 shown in FIG. 1A according to one embodiment. Unlike the conventional garment 200 shown in FIG. 2A, the athletic garment 100 includes the right connective segment 104 and left connective segment 106 that overlay with the right and left armpit areas of the athlete, respectively. For purposes of illustration and clarity, not all components of the athletic garment 100 shown in FIG. 1A are also shown in FIG. 3A.

FIG. 3B is a diagram of the athlete shown in FIG. 3A with raised arms according to one embodiment. The right connective segment 104 and left connective segment 106 compensate for the motion as the athlete raises the athlete's arms. For example, since the connective segments may be composed of an elastic material, for a given elastic deformation of the right connective segment 104, the right arm segment 130 is capable of a threshold movement without causing reciprocal movement of the sensors 114 and 116. Similarly, for a given elastic deformation of the left connective segment 106, the left arm segment 132 is capable of a threshold movement without causing reciprocal movement of the sensors 114 and 116. As another example, for a given elastic deformation of the right connective segment 104, the torso segment 128 (or the left arm segment 132) is capable of a threshold movement without causing reciprocal movement of the sensors on the right arm segment 130 (though not shown in FIG. 3B, for example, see sensors 118, 144, and 148 shown in FIGS. 1A-B). The thresholds of movements may vary for different segments of an athletic garment 100.

As another example way to describe the movement compensation of the connective segments, the connective segments allow the arm segments of the athletic garment 100 to rotate to a given degree without shifting the location of sensors on the torso segment 128 of the athletic garment 100. In particular, the right connective segment 104 includes a reference point 300. The athlete wearing the athletic garment 100 can rotate the athlete's right arm about an axis intersecting the reference point 300, for instance, the axis is orthogonal to the frontal plane (“coronal plane”) of the body of the athlete. In one embodiment, for a given angle 310 of rotation of the right arm segment 130 about the axis, the right arm segment 130 does not cause reciprocal movement of the torso segment 128. Thus, as shown by the dotted lines in FIGS. 3A-B, the sensors 114 and 116 on the torso segment 128 do not shift in location as the athlete's arms are raised. It should be noted that the angle 310 in FIG. 3B is shown to illustrate one example; in other embodiments, the angle that the right arm segment 130 can rotate about the axis without shifting the sensors 114 and 116 may be greater than the angle of approximately 60 degrees shown in FIG. 3B.

### III. Example Athletic Garment With Multiple Layers

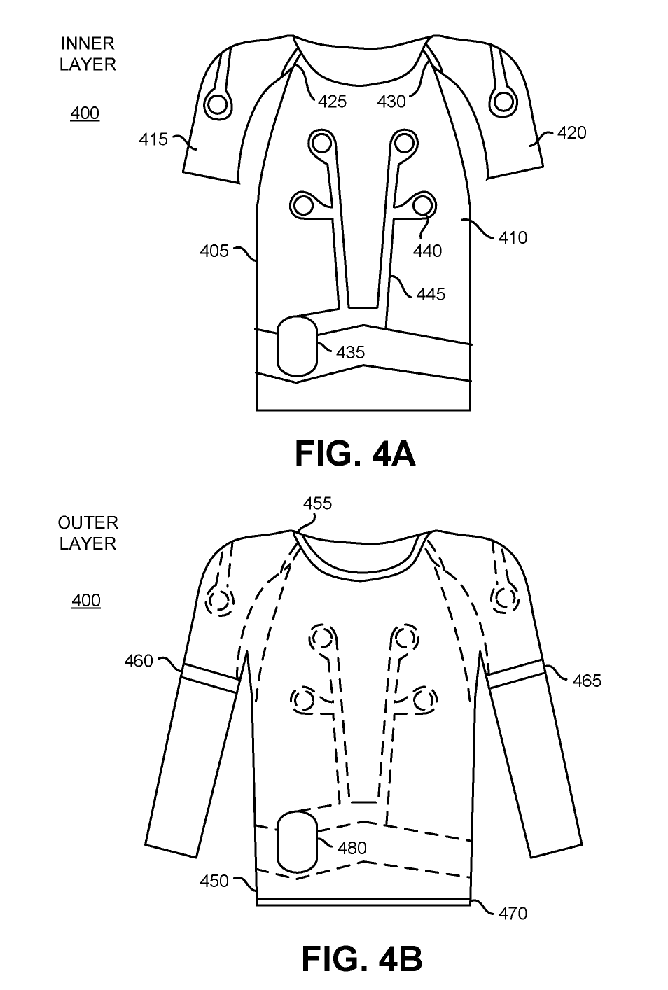

FIG. 4A shows an inner layer 405 of an athletic garment 400 according to one embodiment. The athletic garment 400 also includes an outer layer 450 shown in FIG. 4B and further described below. The inner layer 405 includes one or more sensors 440 electrically coupled via conductive threads 445 to the mount 435, and are substantially the same as the sensors, conductive thread, and mount of the athletic garment 100 shown in FIG. 1A. Also, the right connective segment 425 and left connective segment 430 couple the right arm segment 415 and the left arm segment 420 to the torso segment 410, respectively.

The right connective segment 425 and the left connective segment 430 of the inner layer 405 are different than the right connective segment 104 and the left connective segment 106 of the athletic garment 100. In particular, the connective segments of the athletic garment 100 overlay the armpit area of an athlete wearing the athletic garment 100. In contrast, the connective segments of the inner layer 405 couple the right arm segment 415 and left arm segment 420 of the inner layer 405 to the torso segment 410 without necessarily overlaying the armpit area of the athlete. Rather, in some embodiments, the inner layer 405 exposes the armpit areas of the athlete, and the right connective segment 425 and the left connective segment 430 function as a “hinge” between the arm segments 415 and 420 and the torso segment 410.

The right connective segment 425 and the left connective segment 430 may be composed at least of an elastic material or another material that is the same (or similar to) a material of the torso segment 410, right arm segment 415, or left arm segment 420. Further, one or more conductive threads 445 of the inner layer 405 may be routed from the torso segment 410 to the right and left arm segments 415 and 420 via the right connective segment 425 and the left connective segment 430. In an embodiment where the connective segments are composed of an elastic material, the connective segments may include conductive threads that are configured in a sinusoidal pattern, a “zigzag” pattern, or any other suitable configuration to facilitate expansion or contraction resulting from stretching of the connective segments.

Similar to the movement compensation of the athletic garment 100 described above with reference to FIGS. 3A-B, the inner layer 405 of the athletic garment 400 also compensates for motion of the arms of an athlete wearing the athletic garment 400. As an example, as the athlete raises the arms of the athlete, the right and left connective segments 425 and 430 allow the right arm segment 415 and left arm segment 420 to translate or rotate (e.g., to a given threshold) relative to the torso segment 410 without shifting the location of the sensors on the torso segment 410. By reducing the amount of material overlaying the armpit area (or any other joint area of the body such as the groin area), the athletic garment 400 provides additional mobility to an athlete wearing the athletic garment 400, e.g., particularly for upper body movements in the use case of athletic garment 400. Further, though the material overlaying the armpit area is reduced the athletic garment 400 still enables conductive threads to electrically couple sensors to a mount 102 across the torso segment, left arm segment, and right arm segments (or any other segments such as a leg segment and waist segment for athletic garments worn on the lower body) via connective segments.

FIG. 4B shows an outer layer 450 overlaid on the inner layer 405 of the athletic garment 400 shown in FIG. 4A according to one embodiment. For purposes of illustration, FIG. 4B includes dotted lines to show the sensors 440, conductive thread 445, and right and left connective segments 425 and 430 of the inner layer 405 hidden underneath. The dotted lines also show the armpit areas exposed by the inner layer 405. In embodiments where the outer layer 450 is composed of an opaque material, components of the inner layer 405 may not be visible from a view outside of the athletic garment 400. The outer layer 450 also includes a mount interface 480 for a processing unit configured to be coupled to the mount 435. For example, the mount interface 480 is a connector, port, hole, or any other suitable interface in the outer layer 450 that provides access to the mount 435.

The outer layer 450 can be coupled to the inner layer 405 of the athletic garment 400 at one or more points, in some embodiments. For example, the outer layer 450 is coupled to the inner layer 405 at the collar area 455, right sleeve area 460, left sleeve area 465, or waist area 470 of the athletic garment 400, any combination of thereof, or any other suitable area of the athletic garment such as the shoulder or chest area. In some embodiments, to facilitate the movement compensation of the athletic garment 400, the outer layer 450 is not attached to at least a portion of the inner layer 405, such as the portion of the torso segment 410 including one or more of the sensors 440. Thus, an athlete wearing the athletic garment 400 can move the athlete's arms to a given extent (e.g., corresponding to a particular elastic deformation or rotation of segments of the athletic garment 400) without shifting the locations of sensors of the inner layer 450 relative to target portions of the athlete's body (e.g., muscle group areas).

The outer layer 450 may be composed of a compressive material. The compressive material may be internal to the outer layer 450 or the athletic garment 400, in some embodiments. Further, the outer layer 450 may include one or more elastic bands at the collar area 455, right sleeve area 460, left sleeve area 465, or waist area 470. Thus, the outer layer 450 provides stability to the athletic garment 100 by compressing against the inner layer 405. Accordingly, the overall position of the inner layer 405 relative to the athlete wearing the athletic garment 400 is maintained at least due to the outer layer 450. In some embodiments, the outer layer 450 is not physically coupled to the inner layer 405 and uses compression or elastic bands to maintain its relative position to the inner layer 405.

Furthermore, the outer layer 450 includes material to cover armpit areas of an athlete wearing the athletic garment 400 because the inner layer 405 exposes the armpit areas, in some embodiments. The outer layer 450 may also include connective segments similar to the right and left connective segments 104 and 106 of the athletic garment 100. For example, the outer layer 450 includes connective segments composed of elastic material that overlay the armpit areas of the athlete.

### IV. Example Sleeveless Garments

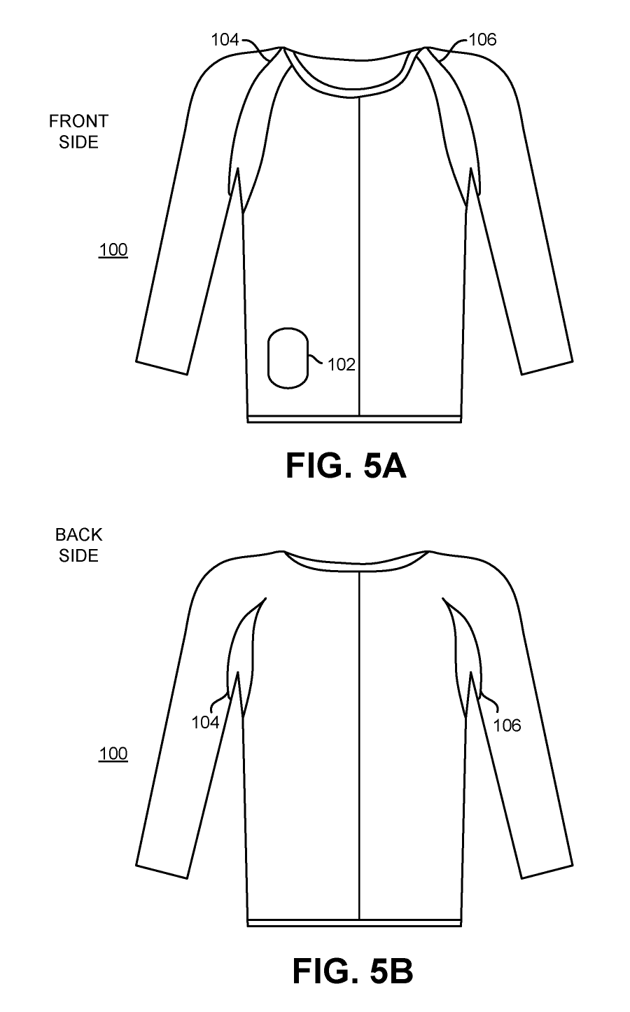

FIG. 5A shows an outside view of the front side of the athletic garment 100 shown in FIG. 1A according to one embodiment. FIG. 5B shows an outside view of the back side of the garment shown in FIG. 1A according to one embodiment. Since the sensors and conductive threads of the athletic garment 100 are on the inside of the athletic garment 100, the sensors and conductive threads are not visible from the outside view of the athletic garment 100 (e.g., composed of an opaque material), in some embodiments.

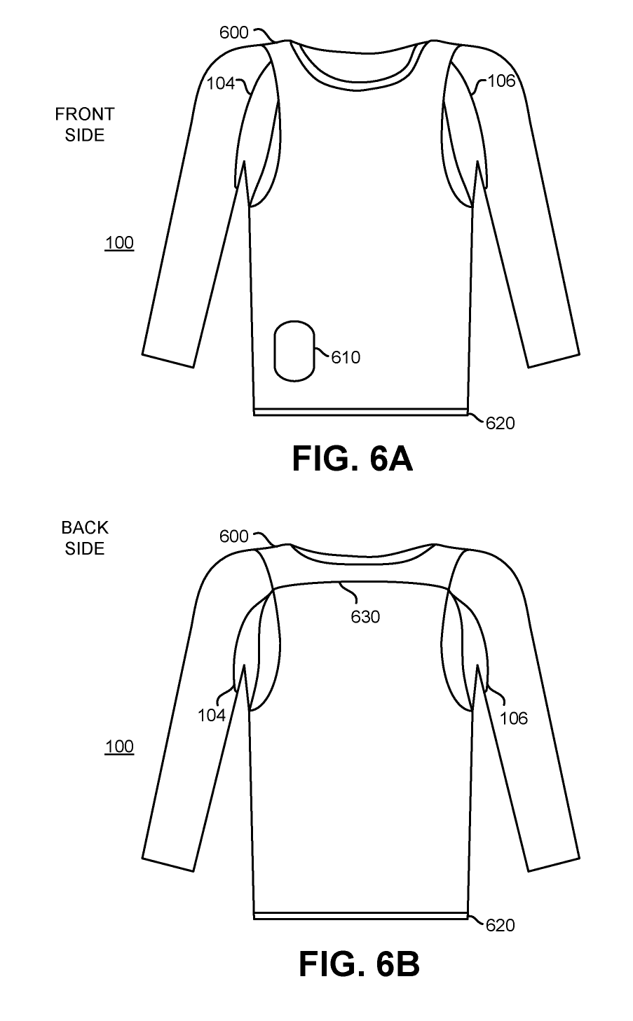

FIG. 6A shows a front side of a sleeveless garment 600 overlaid on the athletic garment 100 shown in FIG. 1A according to one embodiment. FIG. 6B shows a back side of the sleeveless garment 600 overlaid on the athletic garment 100 shown in FIG. 6A according to one embodiment. The sleeveless garment 600 includes a mount interface 610 substantially the same as the mount interface 480 described above with reference to FIG. 4B. The mount interface 610 can be aligned with the mount 102 of the athletic garment 100. Further, the sleeveless garment 600 includes one or more elastic straps (e.g., a compression belt) such as the elastic strap 620 that overlay with the waist of an athlete wearing the sleeveless garment 600 or with the elastic band 108 of the athletic garment 100.

Similar to the outer layer 450 shown in FIG. 4B, the sleeveless garment 600 may be composed at least of compressive material that maintains the position of the athletic garment 100 relative to the body of the athlete. Further, since the sleeveless garment 600 does not have arm segments (i.e., is sleeveless), motion of the athlete's arms does not significantly impact the position of the sleeveless garment 600 relative to the torso of the athlete. Thus, the sensors of the athletic garment 100 will experience minimal shifting in location, relative to sensors of the conventional garment 200, as the user performs exercises that involve motions such as raising the arms of the athlete, for example. The sleeveless garment 600 may also include aesthetic features such as the line 630 that aligns with the top of the right and left connective segments 104 and 106 on the back side of the athletic garment 100.

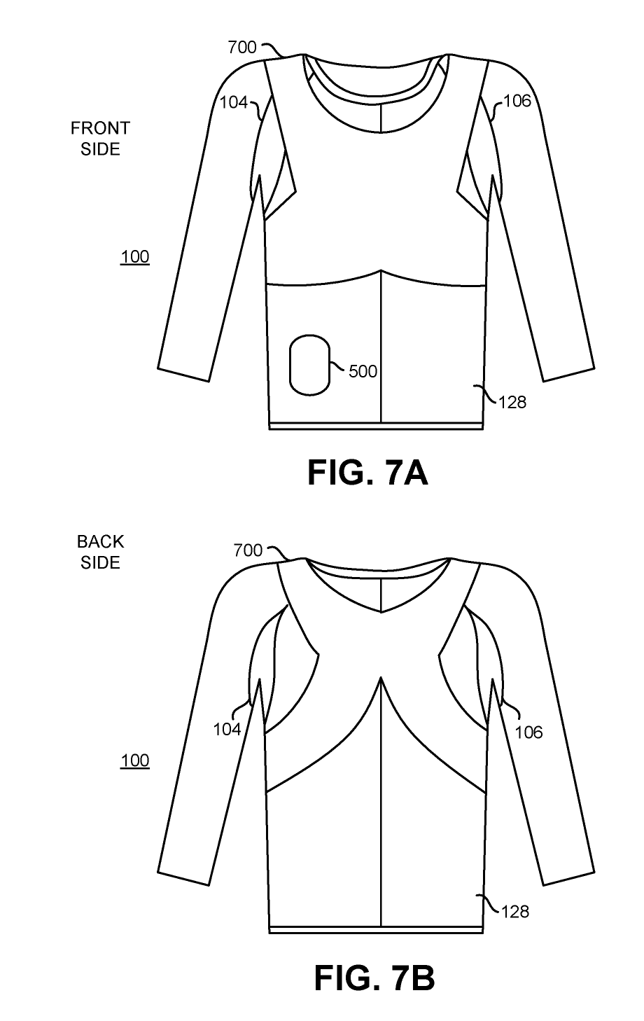

FIG. 7A shows a front side of another sleeveless garment 700 overlaid on the athletic garment 100 shown in FIG. 1A according to one embodiment. FIG. 7B shows a back side of the sleeveless garment 700 overlaid on the athletic garment 100 shown in FIG. 7A according to one embodiment. The sleeveless garment 700 is substantially the same as the sleeveless garment 600 shown in FIGS. 6A-B, though the sleeveless garment 700 has a different form factor. In particular, the sleeveless garment 700 exposes a greater portion of the abdominal area of the torso segment 128 of the athletic garment 100, relative to the sleeveless garment 600. For example, the sleeveless garment 700 is a crop top (e.g., for athletes of any gender) or sports bra styled garment, while the sleeveless garment 600 is a more typical tank top styled garment. In some embodiments, the sleeveless garment 700 or the athletic garment 100 may include one or more zippers to help an athlete put on and take off the sleeveless garment 700 or the athletic garment 100, respectively, e.g., to offset force from a compressive material. Additionally, the athletic garment 100 may also include an adjustable strap to help offset compressive force.

### V. Example Pants Garment

FIG. 8A shows an front view of a pants athletic garment 800 according to one embodiment. FIG. 8B shows a back view of the pants athletic garment 800 shown in FIG. 8A according to one embodiment. The pants athletic garment 800 includes multiple connective segments in the example shown in FIG. 8A-B. In particular, the athletic garment 800 includes right and left connective segments 810A and 810B overlaying a buttocks or groin portion of an athlete's body. The athletic garment 800 includes right and left connective segments 820A and 820B overlaying the knee pit portions of an athlete's body. The athletic garment 800 includes right and left connective segments 830A and 830B overlaying the ankle portions of an athlete's body. In addition, the athletic garment 800 includes a mount 840 for a processing unit configured to be coupled to the mount 840.

The connective segments are coupled to other segments of the athletic garment 800, e.g., leg segments or a torso segment. The athletic garment 800 may include sensors positioned on the leg segments to record physiological data of the athlete's leg muscles. The connective segments minimize shifting of the sensors due to movement of the user's lower body.

### VI. Additional Considerations

Upon reading this disclosure, those of skill in the art will appreciate still additional alternative structural and functional designs through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context unless otherwise explicitly stated.

As used herein, the terms “ comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

What is claimed is:

1. A garment comprising:
a conductive thread coupled within a fabric layer;
a mount that makes electrical contact with the conductive thread;
a plurality of sensors configured to generate physiological data of an athlete wearing the garment and provide the physiological data via the conductive thread to a processing unit coupled to the mount;
a torso segment including a first subset of the plurality of sensors, the torso segment composed of at least a first material having a first elastic modulus;
a connective segment composed of at least a second material having a second elastic modulus less than the first elastic modulus, the connective segment coupled to the torso segment and configured to overlay an armpit of the athlete wearing the garment; and
an arm segment including a second subset of the plurality of sensors, the arm segment coupled to the connective segment.

2. The garment of claim 1, wherein, for a given elastic deformation of the connective segment, (i) the arm segment is capable of a first threshold movement without causing reciprocal movement of the first subset of the plurality of sensors and (ii) the torso segment is capable of a second threshold movement without causing reciprocal movement of the second subset of the plurality of sensors.

3. The garment of claim 1, wherein the connective segment is further composed of at least a third material having a third elastic modulus greater than the second elastic modulus.

4. The garment of claim 1, further comprising an elastic strap coupled to the torso segment.

5. The garment of claim 1, wherein the mount is coupled to the torso segment.

6. The garment of claim 1, wherein the mount is coupled to the arm segment.

7. The garment of claim 1, wherein the torso segment further comprises a second connective segment composed of at least the second material, the second connective segment configured to overlay a spine of the athlete wearing the garment.

8. A garment comprising:
an inner layer including:
a conductive thread;
a mount that makes electrical contact with the conductive thread;
a plurality of sensors configured to generate physiological data of an athlete wearing the garment and provide the physiological data via the conductive thread to a processing unit coupled to the mount;
a first torso segment including a first subset of the plurality of sensors;
a first connective segment coupled to the first torso segment; and
a first arm segment including a second subset of the plurality of sensors, the first arm segment coupled to the first connective segment.

9. The garment of claim 8, wherein the first connective segment includes a reference point, and wherein, for a given range of rotation about an axis intersecting the reference point of the first arm segment relative to the first torso segment, (i) the first arm segment is capable of a first threshold movement without causing reciprocal movement of the first subset of the plurality of sensors and (ii) the first torso segment is capable of a second threshold movement without causing reciprocal movement of the second subset of the plurality of sensors.

10. The garment of claim 8, further comprising an outer layer coupled to the inner layer at one or more of: a portion of the arm segment, a first portion of the torso segment overlaying a neck area of the athlete wearing the garment, or a second portion of the torso segment overlaying a waist area of the athlete wearing the garment.

11. The garment of claim 10, wherein at least part of the outer layer is not coupled to at least part of the inner layer, and wherein the outer layer is composed of at least a compressive material.

12. The garment of claim 8, further comprising an outer layer comprising:
a second torso segment overlaying the first torso segment, the second torso segment composed of at least a first material having a first elastic modulus;
a second connective segment coupled to the second torso segment, the second connective segment composed of at least a second material having a second elastic modulus less than the first elastic modulus, the second connective segment configured to overlay an armpit of the athlete wearing the garment; and
a second arm segment overlaying the first arm segment, the second arm segment coupled to the second connective segment.

13. The garment of claim 12, wherein the second connective segment is further composed of at least a third material having a third elastic modulus greater than the second elastic modulus.

14. The garment of claim 8, wherein the outer layer is not physically attached to the inner layer.

15. A garment comprising:
a conductive thread coupled within a fabric layer;
a first segment composed of at least a first material having a first elastic modulus;
a connective segment composed of at least a second material having a second elastic modulus less than the first elastic modulus, the connective segment coupled to the first segment and configured to overlay a portion of an athlete wearing the garment; and
a second segment coupled to the connective segment.

16. The garment of claim 15, further comprising:
a mount that makes electrical contact with the conductive thread; and
a plurality of sensors configured to generate physiological data of the athlete wearing the garment and provide the physiological data via the conductive thread to a processing unit coupled to the mount, the first segment including a first subset of the plurality of sensors, the second segment including a second subset of the plurality of sensors.

17. The garment of claim 16, wherein, for a given elastic deformation of the connective segment, (i) the second segment is capable of a first threshold movement without causing reciprocal movement of the first subset of the plurality of sensors and (ii) the first segment is capable of a second threshold movement without causing reciprocal movement of the second subset of the plurality of sensors.

18. The garment of claim 15, wherein the connective segment is further composed of at least a third material having a third elastic modulus greater than the second elastic modulus.

19. The garment of claim 15, further comprising an elastic strap coupled to the first segment.

20. The garment of claim 15, wherein the first segment further comprises a second connective segment composed of at least the second material, the second connective segment configured to overlay another portion of the athlete wearing the garment.
